(19) United States
(12) Patent Application Publication
(10) Pub. No.: US 2007/0160549 A1
(43) Pub. Date: Jul. 12, 2007

(54) UV-radiation protectant compositions

(76) Inventors: Sheri Anne Hunt, Memphis, TN (US); Kevin C. Fowler, Millington, TN (US); Thomas A. Meyer, Germantown, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/642,492

(22) Filed: Dec. 20, 2006

Related U.S. Application Data
(60) Provisional application No. 60/752,638, filed on Dec. 21, 2005.

(51) Int. Cl. A61K 8/49; A61K 8/40; A61K 8/29; A61K 8/27

(52) U.S. Cl. 424/59

(57) ABSTRACT

A substantially aqueous composition comprising one or more particle encapsulated sunscreen active agents, at least one volatile additive, and at least one UV-radiation scattering agent, whereby the composition provides an SPF greater than 30.

### CROSS REFERENCE TO RELATED APPLICATION

This application is a non-provisional application that claims priority under 35 U.S.C. §119(e) of provisional application U.S. Ser. No. 60/752,638 filed Dec. 21, 2005, the contents of which are hereby incorporated by reference in their entireties.

### BACKGROUND OF THE INVENTION

It is now generally recognized that exposure to solar radiation can have adverse health consequences, sometimes not appearing until several years following the exposure. Of course, the immediately appearing “sunburn” from an overexposure can itself be a serious acute health problem.

Many products are available to reduce the amount of solar ultraviolet radiation received by the skin during exposure to the sun's rays. Typical product formulations are lotions, creams, ointments or gels containing chemical and/or physical barriers to ultraviolet transmission. These vary considerably in their abilities to protect the skin against the physical and biochemical effects of ultraviolet radiation.

Earlier sunscreening formulations were designed to protect against sunburn from a limited solar exposure period, while transmitting sufficient radiation to permit skin tanning. However, the current focus is on eliminating as much ultraviolet exposure as possible, it being recognized that skin tanning, while esthetically pleasing to some, is a clear indication of tissue damage from overexposure to solar radiation. It has been recently discovered that any amount of unprotected exposure can potentially cause immune system suppression and lead to future health problems, such as skin carcinomas and other dermatological disorders.

The SPF (Sun Protection Factor) rating system has been developed to provide consumer guidance in selecting suitable sunscreens for any given outdoor activity. In general, the SPF number approximately corresponds to the multiple of time during which the properly applied sunscreen will prevent obvious reddening of the skin, over the exposure time that causes unprotected skin to exhibit reddening. Thus, if an SPF 8 sunscreen formulation has been properly applied, a person should be able to remain in the sun without visible effects for eight times the usual unprotected duration. Of course, the duration of unprotected exposure which produces a visible effect on the skin varies from one individual to another, due to differences in their skin cells. Currently popular are high-SPF “sunblocker” products, having SPF values of 30 or greater.

Most of the commercially available sunscreen formulations are not well suited for use by those engaged in strenuous outdoor activities due to the tendency for perspiration from the body to interact with the applied formulation. For example, perspiration, or moisture from other sources, including rain, can cause sunscreen active ingredients and other irritating components of the formulation to enter the eyes and cause discomfort. It is also frequently detrimental, particularly in activities such as tennis or golf which require a reliable grip on equipment, to have an applied sunscreen formulation remain lubricious after application or become lubricious when mixed with perspiration or other moisture.

It is advantageous to have a suncare formulation that is waterproof. Waterproof formulations allow the user to engage in activities such as swimming while still being protected against ultraviolet radiation. Hydrophobic materials typically serve as waterproofing agents that impart film forming and waterproofing characteristics to an emulsion. However, there is still a need for products having physical attributes that display improved waterproof performance, and that have a reduction in migration of the formulation across the formulation wearer's skin, as well as providing a limited slip grip performance attribute.

The application of sunscreen actives in the presence of water/sweat onto skin or hair to afford protection from the damaging effects of UV radiation is still an unmet challenge area. Typically, sunscreens are comprised of oily organic chemistries or inorganic hydrophobic oxides that do not easily disperse in water. Application of such compositions to wet or sweat-soaked skin results in wash-off and uneven coverage, diminishing the full effect of the sunscreen actives. Water-soluble sunscreen formulations have been developed to address this need. However, sunscreen formulations comprising water-soluble UV actives typically require additional waterproofing agents, some of which are not easily dispersed in water. Moreover, addition of other inactive ingredients to sunscreen formulations may also negatively affect the feel and ease of application of the sunscreen. Thus, additional formulation efforts are necessary to provide a consumer-friendly formula that will exhibit the now-desired higher sun protection factor.

Increasingly, liposome technology is being investigated as a means of delivering organic sunscreen active compounds in aqueous formulations. For example, U.S. Pat. No. 5,173,303 to Lau et al. describe methods of forming liposomes containing organic soluble material. Although the Lau patent exemplifies the technology only with the organic soluble pesticide DEET, the patent asserts the technology is equally applicable to other organic soluble compounds, such as sunscreen active UV-absorbing compounds. However, as demonstrated below, the Lau patent methods do not allow the production of high SPF sunscreen formulations. U.S. Pat. No. 5,510,120 to Jones et al. describes liposome-containing cosmetic compositions that are said to be useful to deliver sunscreen agents to skin or hair. The Jones patent describes the formation of liposomes comprising additional means, such as binding proteins, polysaccharides, and glycoproteins, for binding to a target location on the skin and/or hair. U.S. Pat. No. 5,605,740 to Finel et al. describes cationic liposomal dispersions that are said to be useful to deliver anti-dandruff and/or sunscreen agents to hair. Finally, U.S. Pat. No. 6,015,575 to Luther et al. describes compounds with UV-absorbing properties that also incorporate structural elements which make the compounds capable of self-organization into bimolecular layers.

However, there remains a need for improved sunscreen formulations that provide lasting high SPF sun protection chemistries on the skin, in a way that is consumer preferred, in a timely single step when the skin is already wet or moist, and is highly resistant to wash off when later contacted by moisture. Further, there is a need for aqueous formulations of UV-absorbing materials which allow for easier application to surfaces, both skin and non-skin surfaces, in need of protection from UV radiation.

As demonstrated herein, lamellar encapsulation of sunscreen actives provides for quantifiable analytical dilution of organic sunscreen mixtures with water (pre-formula capability) and provides easier formulating of sensitive ingredients, such as antioxidants, to minimize the harmful effects to skin and mucous membranes. The use of lamellar encapsulation also provides for prolonged release of cosmetic additives such as fragrance and sensates. Moreover, the use of lamellar compositions as described herein eliminates the need for emulsifiers which may produce an undesirable oily feel, and improves the viscosity of the product which allows for the production of sprayable high SPF products. Finally, lamellar encapsulation provides for the production of low viscosity, high SPF sunscreen formulations.

### SUMMARY OF THE INVENTION

Accordingly, the benefits noted above are provided by the composition and methods of the subject invention.

Thus, the subject invention provides a substantially aqueous composition comprising one or more lamellar encapsulated sunscreen active agents, at least one volatile additive, and at least one UV-radiation scattering agent, whereby the composition provides an SPF greater than 30.

The invention further provides a substantially aqueous composition for topical administration to a subject comprising one or more liposome encapsulated sunscreen active compounds, one or more UV-radiation scattering agents, and one or more cosmetically acceptable volatile additives.

The invention additionally provides a high SPF sunscreen composition comprising one or more lamellar encapsulated sunscreen active compounds in an aqueous dispersion containing one or more cosmetically acceptable volatile additives and one or more UV-radiation scattering agents.

The invention also provides a substantially waterproof, high SPF sunscreen composition comprising one or more lamellar encapsulated sunscreen active compounds in an aqueous dispersion containing one or more cosmetically acceptable volatile additivies, one or more UV-radiation scattering agents, and one or more skin anchoring components, wherein the composition provides an SPF on wet skin greater than the static SPF on dry skin.

The invention further provides a liquid cosmetic composition for topical application to skin and/or hair comprising lamellar particles encapsulating at least one cosmetically effective benefit agent, a substantially aqueous continuous phase, one or more UV-radiation scattering agents and one or more cosmetically acceptable volatile additives, wherein the cosmetic composition provides a high SPF sunscreen.

The invention further provides a method of preventing erythema in a subject comprising topically applying to the subject's skin or hair a substantially aqueous composition comprising particles encapsulating at least one cosmetically effective benefit agent, a substantially aqueous continuous phase, and one or more cosmetically acceptable volatile additives, wherein the composition comprises a high SPF sunscreen.

The invention also provides a method of preventing photoaging of an object comprising applying to the surface of the object a substantially aqueous composition comprising particles encapsulating a UV-absorbing agent in a substantially aqueous continuous phase, one or more volatile additives, and one or more UV-radiation scattering agents.

The invention further provides a method of preventing photoaging of skin comprising applying to skin that will subsequently be exposed to UV radiation, the composition of the invention.

The invention further provides a kit comprising a physiologically acceptable, dissolvable matrix comprising one or more lamellar encapsulated sunscreen active agents and one or more UV-radiation scattering agents and further comprising instructions to dissolve the matrix in a combination of water and at least one volatile additive so as to form the composition of the invention.

The invention also provides a kit comprising a plurality of zones, one of said zones comprising a mixture comprising one or more lamellar encapsulated sunscreen active agents and one or more UV-radiation scattering agents and another of said zones comprising at least one volatile additive, whereby the zones are physically separated from one another.

The invention further provides a kit comprising a non-woven hydrophobic material comprising one or more lamellar encapsulated sunscreen active agents and one or more UV-radiation scattering agents and further comprising instructions to contact the material in a combination of water and at least one volatile additive so as to form the composition of the invention.

The invention further provides a kit comprising a non-woven hydrophilic material comprising one or more lamellar encapsulated sunscreen active agents and one or more UV-radiation scattering agents and further comprising instructions to contact the material in a combination of water and at least one volatile additive so as to form the composition of the invention.

These and other advantages of the invention will be apparent to those of ordinary skill in the art from in the following description.

### DETAILED DESCRIPTION OF THE INVENTION

Unless otherwise defined herein, names given to chemical substances herein generally are either accepted chemical names, or are trade organization or regulatory agency approved names such as CTFA Adopted Names as listed in J. A. Wenninger et al., Eds., CTFA International Cosmetic Ingredient Dictionary, Eighth Ed., The Cosmetic, Toiletry and Fragrance Association, Washington, D.C., 2000.

The term “percent by weight” as used herein means the percent by weight of the ingredient per weight of the overall formulation.

### A. ENCAPSULATION OF BENEFIT AGENTS

The compositions of the invention are unique in providing high SPF aqueous formulations that are not limited to high viscosity formulations. As used herein, the term “high SPF” refers to a SPF rating of at least 30, in particular SPF ratings up to 35, up to 40, up to 45, or up to 50 or higher. The formulations disclosed herein can be produced at low viscosity, not previously possible with prior art high SPF aqueous formulations, which typically incorporate high loads of sunscreen actives and comparatively large amounts of emulsifier to form useful solutions. The presence of these extra agents ultimately produce final compositions that are thick, oily-feeling formulations, which are not preferred by consumers. Aqueous formulations are more versatile in their use, allowing in particular the packaging of sunscreen compositions into sprays and lighter creams. The compositions of the invention achieve these results by encapsulation of the organic soluble sunscreen actives and cosmetic additives, referred to herein collectively or individually as “benefit agents” or the “burden”, within microparticle or nanoparticle structures, in particular lamellar-based structures, such as liposomes, which are then dispersed in aqueous media, primarily water.

In the preferred particles for the practice of this invention, lamellar-based structures, in particular liposomes, are used and the benefit agent may be entrapped in, adsorbed on, or absorbed into the particles.

Methods of making lamellar structures for encapsulation of active ingredients are well known in the art, see e.g., U.S. Pat. Nos. 5,173,303, 5,510,120, 5,605,740, 6,015,575, and references cited there in. Liposomes may be prepared from those surface active materials which are known for the purpose; examples are given in J H Fendler, “Membrane Mimetic Chemistry” (Wiley-Interscience, New York, 1982) and in J N Weinstein and J D Leserman, Pharmac, Ther., 1984 24 207-233. Among the materials most commonly used are phospholipids from natural sources such as lecithin from egg or soya, and synthetic analogues such as L-α-dipalmitoyl phosphatidylcholine (DPPC). In a preferred embodiment, soy lecithin is used as the phospholipids. Charged phospholipids such as phosphatidyl serine are often incorporated in liposomes to improve colloidal stability.

Techniques for preparation of liposomes are also described in G. Gregoriadis, “Liposome Technology—Vol 1”, (CRC Press, 1984) and in PR Cullis et al., “Liposomes—from Biophysics to Therapeutics”, Chapter 5, (Ed. M J Ostro, Marcel Dekker, New York, 1987). Such techniques include sonication (in an ultrasonic bath) of a phospholipid dispersion and reverse phase evaporation, or “extrusion” under pressure through very fine passages such as provided by polycarbonate membranes.

One method for producing liposomes is described in U.S. Pat. No. 5,173,303, which describes a four step process for producing soy lecithin-based liposomes. The process comprises: dissolving sodium salt of pyrithione in deionized water and allowing to stir at ambient temperature; slowly adding the solubilized sodium pyrithione to hydroxylated lecithin, during which time the sample is slowly polytroned; adding the burden to the sodium pyridinethione lecithin solution and mixing while polytroning; with further addition of deionized water; and finally processing the pyridinethione-lecithin-burden suspension through a microfluidizer.

The use of liposome encapsulated sunscreen allows for high levels of burden loading in concentrated preformula, for example up to 45-60% by weight with only 7-12.5% soybean lecithin. The preformula is then typically diluted with water using low shear mixing to yield the resulting desired final formula. The diluting phase may contain other cosmetic actives described herein, such as preservatives, fragrance, cryoprotectants and film formers. The final formulation for a single relationship of UV actives typically comprises discrete lamellae enveloped burden with approximately 1.1-1.5 bilayers as determined by quantified using phosphorous NMR. (Frohlich, M.; Brecht, V.; Peschka-Suss, R. Parameters influencing the determination of liposome lamellarity by ³¹P-NMR. Chemistry and Physics of Lipids, 2001, 109,103-112). As will be recognized by those of ordinary skill in the art, additional oil/organic soluble materials to be incorporated in the final formulation will be added during formation of the lamellar preformula concentrate, whereas water soluble materials will be added to the aqueous continuous phase used for dilution. Examples of such additional materials are discussed in detail below.

In certain preferred embodiments, the compositions of the invention comprise food grade lecithin to form liposomes containing natural surface anchoring means, in particular the ability to anchor to skin and hair. In other embodiments, the compositions of the invention may further comprise an additional anchoring means which enhances the surface binding properties of the compositions, in particular binding to skin or hair. Such anchoring means are described in U.S. Pat. No. 5,510,120. More generally, any molecule that has an affinity for skin and also an affinity for the lamellar structure but which does not disrupt or degrade the lamellar structure would be useful as an anchoring agent. Such molecules that provide interactions such as electrostatic or hydrophobic interactions with skin would be useful. In other embodiments, chelating agents such as triethylenetetraamine hexaacetic acid (TTHA), ethylenediamine tetraacetic acid (EDTA), diethylenetriamine pentaacetic acid (DTPA), glycol ether diamine tetraacetic acid (GEDTA) and the like can be used. In additional embodiments, the anchoring means is specific for the surface at the selected target. For example, an anchor may allow for use in a topical formulation to be applied to skin or hair will allow for location on the skin and hair not effect binding to organic surfaces at other target sites. The means for binding may be a molecule which binds specifically to a microorganism present at the target site such as molecules having strong affinity for a surface at said target, for example, specifically binding proteins, polysaccharides, glycoproteins, phospholipids, glycolipids, lipoproteins or lipopolysaccharide. A further example includes using a lectin bound to the outer surface of the particles, such as wheat germ agglutinin (WGA) and concanavalin A (ConA).

In a preferred embodiment, the anchoring mechanism comprises polyvalent metal salts of pyrithione, also known as 1-hydroxy-2-pyridinethione; 2-pyridinethiol-1-oxide; 2-pyridinethione; 2-mercaptopyridine-N-oxide; pyridinethione; and pyridinethione-N-oxide, as described in U.S. Pat. No. 6,849,584. Preferred pyridinethione salts include those formed from heavy metals such as zinc, tin, cadmium, magnesium, aluminum and zirconium, preferably zinc, more preferably the zinc salt of 1-hydroxy-2-pyridinethione, which is also referred to herein as “zinc pyridinethione”, “ZPT” or “zinc omidine”. Salts formed from other cations, such as sodium, may also be suitable. Production and use of pyridinethione salts, which are typically incorporated in cosmetic products as anti-dandruff agents and antibacterial or antimicrobial agents, are described in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. In the practice of the current invention, the optional skin anchoring agents can be present in amounts up to about 0.1% (wt/wt), preferably in the range of about 0.01 to about 0.075% (wt/wt) and most preferably in the range of about 0.025 to about 0.05% (wt/wt).

### B. COMPOSITION COMPONENTS

The final formulated products of the invention will comprise lamellar structures, which contain generally oil-soluble, organic sunscreen compounds, in a substantially aqueous continuous phase. Those of ordinary skill in the art will recognize that the following description of various components that the formulations may comprise will include both primarily aqueous-soluble as well as primarily oil-soluble components. It will be recognized that the primarily oil-soluble components will be contained within the lamellar structure in the final formulation and that the primarily aqueous-soluble additional components will be contained in the aqueous continuous phase.

For purposes of the present invention, a “sunscreen active agent” or “sunscreen active” shall include all of those materials, singly or in combination, that are regarded as acceptable for use as active sunscreening ingredients based on their ability to absorb UV radiation. Such compounds are generally described as being UV-A, UV-B, or UV-A/UV-B active agents. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the US but are allowed in formulations sold outside of the US include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetra methylbutyl phenol, and bis-ethylhexyloxyphenol methoxyphenyltriazine, 4-methylbenzylidenecamphor, and isopentyl 4-methoxycinnamate.

However, as the list of approved sunscreens is currently expanding, those of ordinary skill will recognize that the invention is not limited to sunscreen active agents currently approved for human use but is readily applicable to those that may be allowed in the future. The compositions described herein are designed for use with all organic-soluble molecules that will benefit from application in an aqueous composition. In addition, the compositions can comprise micronizable, aqueous dispersable inorganics such titanium and zinc compounds, in particular TiO₂ and ZnO.

In particular embodiments, the sunscreen active agents can include homosalate, available under the trade names Uniderm Homsal (Universal Preserv-A-Chem) and Neo Heliopan HMS (Symrise); benzophenone-3, available under the trade names Escalol 567 (International Specialty Products), Uvinul M-40 (BASF) and Uvasorb MET/C (3V Inc.); octisalate, available under the trade names Neo Heliopan OS (Symrise) and Escalol 587 (International Specialty Products); octacrylene, available under the trade names Uvinul N-539-T (BASF) and Neo Heliopan 303 (Symrise); octinoxate available under the trade names Parsol MCX (DSM Nutritional Products, Inc.) and Uvinul MC 80 (BASF); avobenzone available under the trade names Parsol 1789 (DSM Nutritional Products) and Uvinul BMBM (BASF); ethylhexyl triazone available under the trade name Uvinul T 150 (BASF); bis-ethylhexyloxyphenol methoxyphenyltriazine available under the tradename Tinosorb S (Ciba Specialty Chemicals, Inc.), and methylene bis-benzotriazolyl tetramethylbutylphenol available under the trade name Tinosorb M (Ciba Specialty Chemicals, Inc.); and terephthalylidene dicamphor sulfonic acid sold under the name Mexoryl SX (L'Oreal). Certain embodiments of the invention can contain mixtures of one or more sunscreen actives, inducing mixtures of those recited above.

It is typical to use combinations of two or more sunscreen ingredients in a formulation, to achieve higher levels of ultraviolet absorption or to provide useful absorption over a wider range of ultraviolet wavelengths than can be the case with a single active component. Several other sunscreen active ingredients are accepted for use in other countries and are also considered to be within the scope of the present invention.

The compositions of the invention may also include materials that nonetheless increase the SPF of the final solution by such mechanisms as UV radiation scattering and dispersion. Such materials are referred to herein as “UV-radiation scattering agents” and comprises materials that exhibit UV absorbing activity or no UV absorbing activity. An example of such UV-radiation scattering agents include polymeric materials, such as the product known as SunSpheres™ (Rohm and Haas; Philadelphia, Pa.) which are described by their manufacturer as hollow styrene/acrylates copolymer spheres manufactured by emulsion polymerization. The polymer spheres are said to raise SPF values across the UVA and UVB region by dispersing and/or scattering the incident UV radiation throughout the film of sunscreen present on a surface, such as human skin. It is understood that the spheres cause less UV radiation to penetrate into the skin by redirecting the radiation towards the UV-absorbing sunscreen actives in the sunscreen formulation, where the radiation reacts with the sunscreen active molecules and the energy is dissipated as heat. As used herein, the terms “spheres” or “scattering agents” are not limited by chemical makeup or shape, but comprise any agent that produces the effect of lengthening the path of incident UV radiation, increasing the statistical likelihood that the radiation will contact a sunscreen active molecule, i.e., a UV absorbing active agent. These materials may also include UV absorbing materials that also exhibit scattering properties such as ZnO (examples include Z-Cote™ products available from BASF), TiO₂ (examples include the Solaveil™ products available from Uniqema (New Castle, Del., USA)), compounds such as methylene bis-benzotriazolyl tetramethylbutylphenol, (“Tinasorb™ M” available from Ciba Specialty Chemicals, Inc. (Basel, Switzerland). UV radiation scattering agents are typically present in the formulation in amounts up to about 10% by weight, preferably in ranges of about 0.5% to about 7.0% by weight, in particularly preferred ranges of 3% to about 5% by weight.

As used herein, the term “volatile additive” refers to the a component or components in the formulation that aid the formation of a film of active ingredients on the surface to be protected and quickly evaporate from the surface after application. Such volatile organic solvents include, without limitation, C₁-C₄ straight chain or branched chain alcohol, for example, methanol, ethanol, butanol, and isopropanol, volatile silicone compounds, such as hexamethyldisiloxane, octamethyltrisiloxane, decamethyltetrasiloxane, hexadecamethylheptasiloxane, octamethylcyclotetrasiloxane, decamethylcyclopentasiloxane, tetradecamethylcyclohexasiloxane and volatile aldehydes. Additional examples of each of these are known to those of ordinary skill in the art.

Because the compositions of the invention are substantially aqueous, the volatile additive is present in amounts that would not exceed 50% of the composition. The volatile additive is typically present in an amount up to about 20% by weight of the composition, preferably in an amount up to about 10% by weight, and most preferably in an amount from about 1% by weight up to about 5% by weight. When the composition of the invention comprises a sunscreen to be applied to human skin, the volatile additive should ideally be one that is approved for use in cosmetic compositions. In a preferred embodiment for human sunscreen compositions according to this invention, the volatile additive is ethanol.

As used herein, an “after sun” formulation is defined as a formulation that can be administered after a user has been in the sun for any amount of time that provides a soothing or healing effect that is pleasant to the user. Such a formulation can contain, for instance, aloe vera, vitamins A, C and E, green tea extract, etc.

Compositions of the invention can further comprise so called sunless-tanning or self-tanning compositions, that is, compositions which, when applied to human skin, impart thereto an appearance similar to that achieved by exposing the skin to natural or artificial sunlight. Examples of sunless tanning active agents are described in U.S. Pat. Nos. 6,482,397, 6,261,541, and 6,231,837. Such sunless tanning compositions typically comprise, in addition to an artificial tanning effective amount of a self tanning agent, effective amounts of a composition coloring agent and a cosmetically acceptable carrier adapted for topical application to human skin.

The compositions of the invention can further comprise self tanning agents included generally accepted in the art for application to human skin, and which, when so applied, react therein with amino acids so as to form pigmented products. Such reactions give the skin a brown appearance similar to the color obtained upon exposing it to sunlight for periods of time sufficient to tan the skin. Suitable self tanning agents include, without limitation, alpha-hydroxy aldehydes and ketones, glyceraldehyde and related alcohol aldehydes, various indoles, imidazoles and derivatives thereof, and various approved pigmentation agents. Presently preferred herein as self tanning agents are the alpha-hydroxy aldehydes and ketones. Most preferably, the self tanning agent is dihydroxyacetone (“DHA”). Other suitable self tanning agents include, without limitation, methyl glyoxal, glycerol aldehyde, erythrulose, alloxan, 2,3-dihydroxysuccindialdehyde, 2,3-dimethoxysuccindialdehyde, 2-amino-3-hydroxy-succindialdehyde and 2-benzylamino-3-hydroxysuccindialdehyde.

The compositions of the invention can further comprise skin protectant active agents. Suitable examples include (with preferred weight percent ranges), Allantoin (0.5 to 2 percent); Aluminum hydroxide gel (0.15 to 5 percent); Calamine (1 to 25 percent); Cocoa butter (greater than 50); Cod liver oil (5 to 14 percent); Colloidal oatmeal; Dimethicone (1 to 30 percent); Glycerin (20 to 45 percent); Hard fat (greater than 50); Kaolin (4 to 20 percent); Lanolin (12.5 to 50 percent); Mineral oil (greater than 50 percent); Petrolatum (greater than 30 percent); Sodium bicarbonate; Topical starch (10 to 98 percent); White petrolatum (greater than 30 percent); Zinc acetate (0.1 to 2 percent); Zinc carbonate (0.2 to 2 percent); and Zinc oxide (1 to 25 percent).

The compositions of the invention may further include insect repelling components. The most widely used active agent for personal care products is N,N-Diethyl-m-toluamide, frequently called “DEET” and available in the form of a concentrate containing at least about 95 percent DEET. Other synthetic chemical repellents include dimethyl phthalate, ethyl hexanediol, indalone, di-n-propylisocinchoronate, bicycloheptene, dicarboximide and tetrahydrofuraldehyde. Certain plant-derived materials also have insect repellent activity, including citronella oil and other sources of citronella (including lemon grass oil), limonene, rosemary oil and eucalyptus oil. Choice of an insect repellent for incorporation into the sunscreen emulsion will frequently be influenced by the odor of the repellent. The amount of repellent agent used will depend upon the choice of agent; DEET is useful at high concentrations, such as up to about 15 percent or more, while some of the plant-derived substances are typically used in much lower amounts, such as 0.1 percent or less.

The compositions of the present invention may contain a wide range of additional, optional components which are referred to herein as “cosmetic components”, but which can also include components generally known as pharmaceutically active agents. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present invention. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, SPF boosters, waterproofing agents, and viscosity increasing agents (aqueous and nonaqueous).

In the practice of the invention it is generally preferred to use water which has been purified by processes such as deionization or reverse osmosis, to improve the batch-to-batch formulation inconsistencies which can be caused by dissolved solids in the water supply. The amount of water in the emulsion or composition can range from about 15 percent to 95 weight percent, preferably from about 45 to 75 percent, most preferably from about 60 percent to about 75 percent.

An emollient is an oleaginous or oily substance which helps to smooth and soften the skin, and may also reduce its roughness, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. Preferably, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C₁₂-C₁₅ Alkyl Benzoate sold under the trade name Finsolv TN from Finetex. One or more emollients may be present ranging in amounts from about 1 percent to about 10 percent by weight, preferably about 5 percent by weight. Another suitable emollient is DC 200 Fluid 350, a silicone fluid, available Dow Corning Corp.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C₁₂-C₁₅) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the formulation.

A humectant is a moistening agent that promotes retention of water due to its hygroscopic properties. Suitable humectants include glycerin, polymeric glycols such as polyethylene glycol and polypropylene glycol, mannitol and sorbitol. Preferably, the humectant is Sorbitol, 70% USP or polyethylene glycol 400, NF. One or more humectants can optionally be included in the formulation in amounts from about 1 percent to about 10 percent by weight, preferably about 5 percent by weight.

A dry-feel modifier is an agent which when added to an emulsion, imparts a “dry feel” to the skin when the emulsion dries. Dry feel modifiers can include talc, kaolin, chalk, zinc oxide, silicone fluids, inorganic salts such as barium sulfate, surface treated silica, precipitated silica, fumed silica such as an Aerosil available from Degussa Inc. of New York, N.Y. U.S.A. Another dry feel modifier is an epichlorohydrin cross-linked glyceryl starch of the type that is disclosed in U.S. Pat. No. 6,488,916.

It may be advantageous to incorporate additional thickening agents, such as, for instance, various Carbopols available from the B. F. Goodrich Co. Particularly preferred are those agents which would not disrupt the lamellar structure in the formulation of the final product, such as non-ionic thickening agents. The selection of additional thickening agents is well within the skill of one in the art.

An antimicrobial preservative is a substance or preparation which destroys, or prevents or inhibits the proliferation of, microorganisms in the sunscreen composition, and which may also offer protection from oxidation. Preservatives are frequently used to make self-sterilizing, aqueous based products such as emulsions. This is done to prevent the development of microorganisms that may be in the product from growing during manufacturing and distribution of the product and during use by consumers, who may further inadvertently contaminate the products during normal use. Typical preservatives include the lower alkyl esters of para-hydroxybenzoates (parabens), especially methylparaben, propylparaben, isobutylparaben and mixtures thereof, benzyl alcohol, phenyl ethyl alcohol and benzoic acid, diazolydinyl, urea, chlorphenesin, iodopropynyl and butyl carbamate. The preferred preservative is available under the trade name of Germaben II from Sutton. One or more antimicrobial preservatives can optionally be included in an amount ranging from about 0.001 to about 10 weight percent, preferably about 0.05 to about 1 percent.

An “antioxidant” is a natural or synthetic substance added to the sunscreen to protect from or delay its deterioration due to the action of oxygen in the air (oxidation). They may also reduce oxidation reactions in skin tissue. Anti-oxidants prevent oxidative deterioration which may lead to the generation of rancidity and nonenyzymatic browning reaction products. Typical suitable antioxidants include propyl, octyl and dodecyl esters of gallic acid, butylated hydroxyanisole (BHA, usually purchased as a mixture of ortho and meta isomers), butylated hydroxytoluene (BHT), green tea extract, uric acid, cysteine, pyruvate, nordihydroguaiaretic acid, Vitamin A, Vitamin E and Vitamin C and their derivatives. One or more antioxidants can optionally be included in the sunscreen composition in an amount ranging from about 0.001 to about 5 weight percent, preferably about 0.01 to about 0.5 percent.

“Chelating agents” are substances used to chelate or bind metallic ions, such as with a heterocylic ring structure so that the ion is held by chemical bonds from each of the participating rings. Suitable chelating agents include ethylene diaminetetraacetic acid (EDTA), EDTA disodium, calcium disodium edetate, EDTA trisodium, albumin, transferrin, desferoxamine, desferal, desferoxamine mesylate, EDTA tetrasodium and EDTA dipotassium, or combinations of any of these.

“Fragrances” are aromatic substances which can impart an aesthetically pleasing aroma to the sunscreen composition. Typical fragrances include aromatic materials extracted from botanical sources (i.e., rose petals, gardenia blossoms, jasmine flowers, etc.) which can be used alone or in any combination to create essential oils. Alternatively, alcoholic extracts may be prepared for compounding fragrances. However, due to the relatively high costs of obtaining fragrances from natural substances, the modern trend is to use synthetically prepared fragrances, particularly in high-volume products. One or more fragrances can optionally be included in the sunscreen composition in an amount ranging from about 0.001 to about 5 weight percent, preferably about 0.01 to about 0.5 percent by weight.

A “pH modifier” is a compound that will adjust the pH of a formulation to a more acidic pH value or to a more basic pH value. The selection of a suitable pH modifier is well within the ordinary skill of one in the art.

### C. FORMULATIONS AND PACKAGING

In one embodiment of the invention, the liposomes can be incorporated into aqueous dispersions for direct application to a surface, such as skin. The use of liposomes in certain embodiments of the invention provides a new and unique formula option for high SPF compositions. The liposomes' affinity to the skin minimizes migration of the sunscreen actives laterally along the skin surface and transdermally. This aspect of the invention allows for rinsing of the skin after application of the composition to dry skin, or even application onto a pre-wetted skin surface, without loss of activity. Moreover, as demonstrated herein, the compositions of the invention demonstrate increases the SPF activity of the composition upon exposure to water or moisture.

In a preferred embodiment, the compositions of the invention can be formulated into various products for application to human skin to provide a sunscreen or sunblock. The use of lamellae encapsulation provides for aqueous formulations of high SPF products, allowing for formulations that can be sprayed, wiped or applied in leave-on type formulations from which the aqueous continuous phase evaporates, leaving the liposomes on the surface of the skin. Such products include aerosol and non-aerosol spray formulations and lotions or creams such as skin or hair conditioner products. In a preferred embodiment, the sunscreen compositions of the invention are contained within a pressurized canister containing a valve that releases the composition as a continuous spray when the valve is opened.

The lamellar-encapsulated components may be included in a physiologically acceptable, dissolvable matrix, such as a polymeric matrix, which delivers the lamellar-encapsulated components on dispersion in water and volatile additive. In one such embodiment the liposome encapsulated components can be present as a powder, a dry film, or contained within a woven or non-woven substrate such as a fabric. In such an embodiment, the lamellar encapsulated components and the water and volatile additive components can be contained in a kit that maintains the solid and liquid components in separate zones or compartments for storage, and then provides for mixing and reformulation prior to application. In a preferred embodiment, the kit comprises a packet comprising the separate components in individual wells or compartments separated by a breakable barrier, which barrier can be broken to form a single zone or compartment, to combine the components for reformulation in the single compartment. In a separately preferred embodiment, the compositions of the invention, in particular the lamellar encapsulated sunscreen actives, can also be applied to non-woven hydrophobic or hydrophilic materials which release the materials upon wetting with a water, volatile additive mixture. These hydrophobic or hydrophilic materials can be used to store the compositions of the invention for later application to another surface, such as skin. In this embodiment, lamellar-encapsulated components can be dried, such as by spray drying or lyophilization, and applied to a non-woven substrate from which it is easily re-suspended with water. The release of the lamellar-encapsulated components from the substrate material may be facilitated or enhanced by designing the substrate to bear a charge that is repulsive to the charge on the lamellar surface. The charge may be weakly or strongly repulsive to suit the need for effective delivery of the liposomes to the skin surface during product application.

The articles of the present invention may be packaged individually or with additional articles suitable for providing separate benefits not provided by the primary article, e.g., aesthetic, therapeutic, functional, or otherwise, thereby forming a personal care kit. The additional article of this personal care kit preferably comprises a water insoluble substrate comprising at least one layer and either a cleansing component containing a lathering surfactant or a therapeutic benefit component disposed onto or impregnated into that layer of the substrate of the additional article.

The additional article of the present invention may also serve a functional benefit in addition to or in lieu of a therapeutic or aesthetic benefit. For instance, the additional article may be useful as a drying implement suitable for use to aid in the removal of water from the skin or hair upon completion of a showering or bathing experience.

The articles of the present invention may also comprise one or more chambers or zones or compartments. Such zones or chambers or compartments result from the connection (e.g., bonding) of the substrate layers to one another at various loci to define enclosed areas. These zones or compartments or chambers are useful, e.g., for separating various article components from one another, e.g., the surfactant-containing cleansing component from a conditioning agent. The separated article components which provide a therapeutic or aesthetic or cleansing benefit may be released from the chambers in a variety of ways including, but not limited to, solubilization, emulsification, mechanical transfer, puncturing, popping, bursting, squeezing of the chamber or even peeling away a substrate layer which composes a portion of the chamber.

### D. METHODS

The compositions of the invention provide for the production of compositions that can be applied to surfaces of materials that would benefit from reduced exposure to UV radiation. Those of ordinary skill in the art will easily recognize that the compositions of the invention, because of their ability to effectively absorb broad spectrum UV radiation, and the ease with which they can be formulated, are useful in many methods of prevention and treatment when applied to humans or other animals and are useful for application to any object exposed to sunlight and UV radiation that would suffer consequences of said exposure. For example, the composition can be used in a method of preventing “photoaging” of an object, which is defined herein as damage to an object caused by UV radiation contacting the object. Examples of such photoaging include, fading, browning, cracking, and the like. The formulations of the invention can be incorporated into compositions to be applied to surfaces regularly exposed to sunlight that experience surface degradation due to the UV radiation. Such compositions include, but are not limited to, water- and oil-based paints, stains, dyes, gels, polymer-based sheets and coatings, textiles, and metals. In separately preferred embodiments, the coatings of the present invention can be applied to surfaces as part of the manufacturing process of the materials or can be supplied as “after market” components to applied by a consumer in an as needed basis, such as on days when alerted to elevated UV index.

In addition, when formulated so as to be applied to human and or animal skin, the compositions of the invention can be used in methods of preventing photoaging of skin and methods of preventing erythema.

The invention will be further described by means of the following examples, which are not intended to limit the invention, as defined by the appended claims, in any manner.

### E. EXAMPLES

Examples 1-5 demonstrate incorporation of very high loads of single sunscreen active components or mixtures of sunscreen actives in water dispersions using liposome technology according to the present invention. The following procedure was used for making the liposome-encapsulated sunscreen active concentrates set forth in Examples 1-5.

An oil phase mixture was first established by weighing sunscreen active compounds into a 500 ml beaker. Each sunscreen was added in an amount to achieve the desired weight percentage of sunscreen actives in the final concentrate based on a desired total weight of the concentrate of 100 g. Thus, for Example 1, 21.82 g of Homosalate, 9.1 g of Octisalate, 6.36 g of Oxybenzone and 3.64 g of Octocrylene were added to the beaker To this mixture was added 2.5 g polysorbate 80 and 0.05 g zinc pyrithione. The mixture was then heated to 80 C for 30 minutes or until the solution is clear. Overhead stirring was done continuously. The mixture was removed from the heat and allowed to cool to 60 C (+/−2 C). When cooled, 7.0 g soy lecithin was added along with preservatives, such as benzyl alcohol or parabens, in amounts appropriate for formulations to be left on skin under current FDA regulations. Thus, in Example 1, 0.4 g of methylparaben and 0.2 g of propylparaben were added at this point and in Example 2, 1 g of benzyl alcohol as added. As noted below, in the production of final sunscreen formulations, additional preservative systems were also used but were not added as part of the formation of the liposome concentrate. When avobenzone was included as a sunscreen active in the formulation, it was weighed and added to the cooled mixture at this point. The mixture was then stirred for 15 minutes using an overhead stirrer.

In a separate beaker, a water phase was established by heating to 60 C (+/−2 C) an amount of deionized water to form a total weight of 100 g for the final concentrate when combined with the oil phase. The oil phase was then mixed into the water phase with overhead stirring until homogeneous, forming a crude liposome suspension. The final weight of the combined oil phase and water phase was 100 g, adjusted during cooling with additional deionized water, to form the crude liposome suspension. The crude liposome suspension was then processed according to methods described U.S. Pat. No. 5,173,303 for forming a final liposome concentrate.

### Example 1

The following formulation was prepared according to the method described above to form a liposome concentrate comprising a mixture of sunscreen actives (44.6% by weight of concentrate) featuring an oxybenzone- and paraben-containing formula.

| Ingredients | Percent by Weight |
|---|---|
| Water | 45.29 |
| Homosalate | 21.82 |
| Octisalate | 9.1 |
| Soy Lecithin | 7.0 |
| Oxybenzone | 6.36 |
| Avobenzone | 3.64 |
| Octocrylene | 3.64 |
| Polysorbate 80 | 2.5 |
| Methylparaben | 0.4 |
| Propylparaben | 0.2 |
| Zinc Omadine | 0.05 |

### Example 2

The following formulation was prepared according to the method described above to form a liposome concentrate comprising a mixture of sunscreens (44.6% by weight of concentrate) in a paraben-free formula.

| Ingredients | Percent by Weight |
|---|---|
| Water | 44.89 |
| Homosalate | 21.82 |
| Octisalate | 9.1 |
| Soy Lecithin | 7.0 |
| Oxybenzone | 6.36 |
| Avobenzone | 3.64 |
| Octocrylene | 3.64 |
| Polysorbate 80 | 2.5 |
| Benzyl Alcohol | 1.0 |
| Zinc Omadine | 0.05 |

### Example 3

The following formulation was prepared according to the method described above to form a liposome concentrate comprising sunscreen actives (38.2% by weight of concentrate) that do not include oxybenzone.

| Ingredients | Percent by Weight |
|---|---|
| Water | 51.25 |
| Homosalate | 10.91 |
| Octisalate | 9.10 |
| Soy Lecithin | 7.0 |
| Avobenzone | 5.46 |
| Octocrylene | 12.73 |
| Polysorbate 80 | 2.5 |
| Benzyl Alcohol | 1.0 |
| Zinc Omadine | 0.05 |

### Example 4

The following formulation was prepared according to methods described above to form a liposome concentrate comprising sunscreen (44.6% by weight of concentrate) using sunscreens other than avobenzone.

| Ingredients | Percent by Weight |
|---|---|
| Water | 44.78 |
| Homosalate | 20.0 |
| Octinoxate | 10.0 |
| Oxybenzone | 8.0 |
| Octisalate | 6.67 |
| Soy Lecithin | 7.0 |
| Polysorbate 80 | 2.5 |
| Benzyl Alcohol | 1.0 |
| Zinc Omadine | 0.05 |

### Example 5

The following formulation was prepared according to method described above to form a liposome concentrate comprising a single sunscreen active compound (Octocrylene at 45% by weight of concentrate) in a Paraben-containing formula.

| Ingredients | Percent by Weight |
|---|---|
| Water | 41.85 |
| Octocrylene | 45.0 |
| Soy Lecithin | 7.0 |
| Polysorbate 80 | 2.5 |
| Methylparaben | 0.4 |
| Propylparaben | 0.2 |
| Zinc Omadine | 0.05 |

### Example 6

### SPF Testing

The measurement of in vivo SPF intends to simulate end-user application of a standard applied thickness. The U.S. Food and Drug Administration (FDA) sets out protocols for testing “static” (dry skin) SPF values (21 C.F.R. §352.73) and water resistant or very water resistant values (21 C.F.R. §352.76). All SPF testing described herein was conducted according to the U.S. F.D.A. approved testing protocols. Similar testing protocols are employed through various foreign national and regional certification organizations, such as the European Cosmetic Toiletry and Perfumery Association (“COLIPA”). According to these methods, a biological endpoint (erythema) is used to measure the effect of UV absorbers and blockers. The recommended amount of sunscreen to apply in both FDA and COLIPA in vivo methodologies is 2 mg/cm², or 2 μL/cm² since most sunscreens have a specific gravity of almost unity. The area of application was measured for each subject and then the corresponding amount of sunscreen is measured using a pipette (volume) or weighed by loss. In vivo SPF tests were preformed on at least three (3) subjects in each instance, but repeated only up to the number of times deemed necessary to establish performance of the sunscreen compositions on human skin.

Sunscreen formulations containing the liposome concentrates described in Examples 1-3 above were prepared according to known methods. The amount of sunscreen in the formulation was determined based on the type(s) of sunscreen actives present in the liposome concentrate. The liposomes concentrates were diluted with water to provide levels of sunscreen actives in the final composition that would be expected to yield an SPF of 45 in prior art emulsion systems. Low shear overhead mixers were used to homogeneously distribute the liposomes with volatile additives and scattering agents in the final formulations.

The Sunspheres used in the examples are supplied by the manufacturer (Rohm &Haas) in either a micronized powder or nano-dispersion. The nano-dispersion is used as supplied from the manufacturer using low shear overhead mixers (G.K. Heller Corp. Heavy-Duty Laboratory Stirrer Type M0399015 with G.K. Heller Corp. Series S Motor Controller). The powder required dispersion in water with the high shear mixer prior to addition of the liposome concentrate, using a Gilford homogenizer (Gifford-Ward Eppenbach Homo-Mixer w/ Staco Variable Transformer Model 3PN1010B) set at 70% power for about 15 minutes, followed by low shear mixing of liposome concentrate and volatile additives.

Certain of the sunscreen formulations contain the additional ingredients propylene glycol as a cryoprotection additive and chlorphenesin as an additional preservative, both of which are understood not to contribute to SPF values. The viscosities for all of the tested formulations remain water thin (<500 cps), which is a key feature and advantage of the present invention. Final sunscreen formulations made from the liposome concentrate of Examples 1 and 2 above contained the same amount and type of sunscreen actives in their formulations. The amount of sunscreen in these final sunscreen formulation was also identical to the amount and type of sunscreen actives present in prior art emulsion formulation COPPERTONE® SPF 45. This prior art product was used as a control and tested alongside the experimental liposome formulations in these in vivo tests on the same human subjects and calculated to have an SPF of 45 in these tests. For the sunscreen composition formed with the liposome concentrate of Example 3, the type and amount of sunscreen actives used was calculated to deliver an in vivo SPF 45 based on knowledge of one of skill in the art. The final sunscreen formulations tested were as follows:

TABLE 1

| Formulation Ingredients | Percent by Weight |
|---|---|
| Example 1: | |
| Liposome Concentrate #1 | 54.945 |
| Purified Water | 45.055 |
| Example 2: | |
| Liposome Concentrate #2 | 54.945 |
| Propylene Glycol | 5.0 |
| Chlorphenesin | 0.2 |
| Purified Water | 39.855 |
| Example 2 + Scattering Agent: | |
| Liposome Concentrate #2 | 54.945 |
| Propylene Glycol | 5.0 |
| Sunspheres ™ | 18.1 |
| Chlorphenesin | 0.3 |
| Purified Water | 21.655 |
| Example 2 + Scattering | |
| Agent + Volatile Additive: | |
| Liposome Concentrate #2 | 54.945 |
| Propylene Glycol | 5.0 |
| Sunspheres ™ | 18.1 |
| Chlorphenesin | 0.1 |
| SD-40 Alcohol | 10 |
| Purified Water | 11.755 |
| Example 3: | |
| Liposome Concentrate #3 | 54.945 |
| Propylene Glycol | 5.0 |
| Purified Water | 40.055 |
| Example 3 + Scattering Agent: | |
| Liposome Concentrate #3 | 54.945 |
| Propylene Glycol | 5.0 |
| Sunspheres ™ | 18.1 |
| Purified Water | 21.955 |
| Example 3 + Volatile Additive: | |
| Liposome Concentrate #3 | 54.945 |
| Propylene Glycol | 5.0 |
| SD-40 Alcohol | 5.0 |
| Purified Water | 35.055 |
| Example 3 + Scattering | |
| Agent + Volatile Additive: | |
| Liposome Concentrate #3 | 54.945 |
| Propylene Glycol | 5.0 |
| Sunspheres ™ | 18.1 |
| SD-40 Alcohol | 5.0 |
| Purified Water | 16.955 |

Evaluation

Table 2 shows static SPF results for three different compositions, each composition prepared by diluting the liposome concentrates of Examples 1-3, respectively, with water such that the liposome concentrate comprised 54.945% w/w of the final sunscreen composition.

TABLE 2

| Formula | In Vivo SPF |
|---|---|
| Example 1 | 10.0 |
| Example 2 | 18.0 |
| Example 3 | 12.5 |

The data shows that the static SPF values obtained from human testing fall significantly short of the expected SPF 45. SPF values for sunscreen compositions can be increased by including a scattering agent in the composition with the sunscreen actives.

Table 3 shows the results of static SPF testing of sunscreen formulations prepared from liposome concentrates of Examples 2 and 3, diluted with deionized water, and further comprising Sunspheres™ SPF boosters. The compositions were formulated such that the liposome concentrate comprised 54.945% by weight of the final formulation and the Sunspheres™ comprised 5% by weight of the final formulation, the highest amount recommended by the manufacturer.

TABLE 3

| Formula | In vivo SPF |
|---|---|
| Example 2 + Scattering Agent | 19.7 |
| Example 3 + Scattering Agent | 38.1 |

As shown in Table 3, addition of the scattering agent to the sunscreen formulations formed from liposome concentrates of Examples 2 and 3 increased the SPF of both formulas, as anticipated. However, the values of SPF still remained significantly lower than the expected value of SPF 45. Accordingly, additional in vitro experiments were conducted to investigate the film forming properties of the liposome-encapsulated sunscreens for efficacy, for example by the addition of non-volatile additives such as plantaren. These experiments showed little to no impact on SPF for sunscreen formulations comprising only the liposome encapsulated sunscreens or sunscreen formulations comprising the liposomes and a scattering agent.

However, it was surprisingly discovered that the addition of volatile additives in combination with a scattering agent to the liposome system generated an unexpected boost in SPF. Tables 4 and 5 below show the results of experiments demonstrating the unexpected boost to static SPF values from addition of SD-40 alcohol (ethanol) in compositions comprising liposomes and spheres. The SPF results for each formula in the presence of different additives are provided for comparison purposes.

TABLE 4

| Formula | In Vivo SPF |
|---|---|
| Example 2 | 18.0 |
| Example 2 + Scattering Agent | 19.7 |
| Example 2 + Scattering Agent + | 39.0 |
| Volatile Additive | |

TABLE 5

| Formula | In vivo SPF |
|---|---|
| Example 3 | 12.5 |
| Example 3 + Volatile Additive | 10.5 |
| Example 3 + Scattering Agent | 38.1 |
| Example 3 + Scattering Agent + | 46.0 |
| Volatile Additive | |

These data show that (1) addition of volatile additive by itself provided no boost in SPF; (2) addition of scattering agent by itself boosted SPF, but did not recover to anticipated SPF values calculated for the amount of sunscreens in the formulation; and (3) addition of scattering agent plus volatile additives together provided the highest boost in SPF.

A sunscreen formulation based on the liposome concentrate of Example 2 was then tested according to US FDA protocol for determining very water resistant SPFs. Table 6 below shows the results of the tests. Static SPFs from above are repeated below for comparison to very water resistant SPFs.

TABLE 6

| Formula | Static SPF | VWR SPF |
|---|---|---|
| Example 2 | 18.0 | 14.0 |
| Example 2 + Scattering Agent | 19.7 | 35.0 |
| Example 2 + Scattering Agent + | 39.0 | 58.0 |
| Volatile Additive | | |

The results show that formulas containing liposomes in combination with either scattering agents or scattering agents plus volatile additive yield significantly higher SPF results after being exposed to water relative to sunscreen formulations comprising liposome encapsulated sunscreens only. Thus, another surprising benefit of the present invention is that values of SPF increase after exposure to water, relative to static measurements on skin not exposed to water. This result was also unexpected.

### Example 7

### Finger Dip Test

The following demonstrates the advantages provided by a preferred embodiment of the invention, wherein the liposome composition includes a skin anchoring agent. In the Examples above, zinc omidine, also known as zinc pyrithione, is used as the anchoring agent. U.S. Pat. No. 5,173,303 describes liposome-based compositions for application to skin that contain sodium pyrithione in the water phase. In the present experiments, zinc pyrithione was incorporated into the oil phase and tested in the SPF water resistance test (very water resistant (VWR)) for its ability to maintain high SPF after exposure to water for 80 minutes. In typical sunscreen formulations, maintaining SPF in the VWR testing is achieved by incorporating waterproofing polymers into the formulation, which tend to leave a sticky unpleasant feel on the skin. The presence of liposomes in the present embodiment formulation provides a certain measure of skin anchoring of the final formulation such that waterproofing polymers are not needed. However, it was discovered that the addition of the zinc pyrithione in the oil phase enhances this feature of the invention. Moreover, including the additional skin anchoring agent such as zinc pyrithione in the liposome formulations provides an additional benefit of being able to apply the sunscreen composition to already wet skin and have the sunscreens remain on the skin.

To evaluate these beneficial features of the invention an experimental protocol was designed to quantitate deposition on dry and wet skin. Liposome systems with and without the zinc pyrithione were tested. The following protocol was developed to evaluate liposomes retention with and without zinc pyrithione on skin when a finger, dry or wet, is dipped into an appropriate suspension.

A 20 ml sample of sunscreen formulation was placed into a 30 ml plastic cup. Each finger dip was confirmed to end with the tip of the finger at the center point of the bottom of the cup. A chosen finger was pre-washed with isopropyl alcohol (IPA) to normalize for skin oils and then air dried for 10 minutes. The wet and dry skin formula application was identical except that for the wet skin test the finger was subject to a pre-wetting step, which was omitted for the dry skin test.

For pre-wetting, the finger tip testing area (“dip area”) was dipped into 20 ml tap water in a 30 ml plastic cup for 10 seconds. Only the dip area became wet. The dip area was then inserted into 20 ml of sunscreen formulation for 10 seconds. Excess formulation was removed by dabbing fingertip onto a paper towel three times onto separate areas. The saturated finger was then swirled 30 times inside a plastic cup containing 20 ml of water inside, making sure not to touch the edges during the procedure so as not to dislodge any of the formulation by contact with the cup. The dip area was then inserted into 100 ml IPA and mixed until all the sunscreen was removed from the finger into the IPA. The finger was then inserted into water to wash off excess IPA. A 3 ml sample of the liposome/IPA solution was then added to 7 ml of IPA to dilute to adequate absorbance to satisfy the instrument requirements (Perkin Elmer Lambda 40 UV/VIS Spectrophotometer). The liposome/IPA solution as then assayed by scanning for absorbance from 290 nm to 400 nm, indicating the amount of sunscreen actives.

Under the circumstances of the current protocol, the Beer-Lambert law is obeyed, which expresses the linear relationship between absorbance and concentration of the absorbing chemistries:
A=εbc

wherein A is absorbance, ε is the molar absorbtivity (L/mol·cm), b is the path length of the sample, i.e., the path length of the cuvette in which the sample is contained (cm); and c is the concentration of the compound in solution, (mol/L). Therefore, holding the path length and chemical composition constant, an increase in absorption would be due to increased concentration of the absorbing species present in the solution. Looking at one wavelength, 310 nm, the amount of absoring species left on the skin was quantified after dipping into the solution. Table 7 shows the results of the tests using a sunscreen formulated from the liposome concentrate of Example 2 with scattering agent and volatile additive and zinc pyrithione on one subject. The control formula was the same as the test formula but lacking zinc pyrithione.

TABLE 7

Absorbance at 310 nm

| Skin Application Method | Control | Test Formula |
|---|---|---|
| Dry with water wash | 0.60 | 0.98 |
| Wet with water wash | 0.23 | 0.66 |

As shown in Table 7, the liposome formulations of the invention containing zinc pyrithione show greater skin affinity than the liposome formulations without the zinc pyrithione. This holds true for deposition on pre-wetted skin or dry skin. The natural affinity that liposomes alone have for skin is shown to be enhanced by the presence of a skin anchoring agent in the oil phase, as demonstrated by a reduction in the amount rinsed off after application to wet or dry skin. A boost of 63% was seen for retention of the liposome-containing sunscreen actives applied to dry skin and a boost of 186% was seen on application to pre-wetted skin.

Although certain presently preferred embodiments of the invention have been described herein, it will be apparent to those skilled in the art to which the invention pertains that variations and modifications of the described embodiments may be made without departing from the spirit and scope of the invention. Accordingly, it is intended that the invention be limited only to the extent required by the appended claims and the applicable rules of law.

What is claimed is:

1. A substantially aqueous composition comprising one or more lamellar encapsulated sunscreen active agents, at least one volatile additive, and at least one UV-radiation scattering agent, whereby the composition provides an SPF greater than 30.

2. The composition of claim 1, wherein the lamellar encapsulated sunscreen active agent is a liposome-encapsulated sunscreen active agent.

3. The composition of claim 2, wherein the liposome is formed from food grade soy lecithin.

4. The composition of claim 1, wherein the volatile additive is chosen from the group consisting of C₁-C₄ straight chain or branched chain alcohol, volatile silicone alcohol, C₁-C₄ volatile aldehyde or mixtures thereof.

5. The composition of claim 4, wherein the C₁-C₄ straight chain or branched chain alcohol is chosen from the group consisting of methanol, ethanol, propanol, butanol, and isopropanol.

6. The composition of claim 5, wherein the volatile additive is ethanol.

7. The composition of claim 1 wherein the volatile additive is present in the composition in an amount less than 10% by weight.

8. The composition of claim 1, wherein the volatile additive is present in the composition in an amount between about 1% by weight and about 5% by weight.

9. The composition of claim 1, wherein the UV-radiation scattering agent is chosen from the group consisting of polymer spheres, ZnO, TiO₂, methylene bis-benzotriazolyl tetramethylbutylphenol, and combinations thereof.

10. The composition of claim 9, wherein the polymer spheres comprise styrene/acrylates copolymers.

11. The composition of claim 1, wherein the sunscreen active compound is chosen from the group consisting of para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum, 4-methylbenzylidenecamphor, and isopentyl 4-methoxycinnamate, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, and combinations thereof.

12. A substantially aqueous composition for topical administration to a subject comprising one or more liposome encapsulated sunscreen active compounds, one or more UV-radiation scattering agents, and one or more cosmetically acceptable volatile additives.

13. The composition of claim 12, wherein the volatile additive is chosen from the group consisting of C₁-C₄ straight chain or branched chain alcohol, volatile silicone alcohol, C₁-C₄ volatile aldehyde or mixtures thereof.

14. The composition of claim 13, wherein the volatile additive is ethanol.

15. The composition of claim 14 wherein the volatile organic vehicle is present in the composition in an amount less than 10% by weight.

16. The composition of claim 12, wherein the sunscreen active compound is chosen from the group consisting of para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum, 4-methylbenzylidenecamphor, and isopentyl 4-methoxycinnamate, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, and combinations thereof.

17. The composition of claim 12 further comprising additional cosmetic components.

18. The composition of claim 17, wherein the cosmetic components comprise one or more of self-tanning agents, insect repelling components, skin protectant active agents, absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, propellants, reducing agents, skin bleaching agents, skin-conditioning agents, skin protectants, solvents, foam boosters, hydrotropes, solubilizing agents, nonsurfactant suspending agents, waterproofing agents, and viscosity increasing agents.

19. The composition of claim 12, further comprising zinc pyridinethione.

20. A high SPF sunscreen composition comprising one or more lamellar encapsulated sunscreen active compounds in an aqueous dispersion containing one or more cosmetically acceptable volatile additives and one or more UV-radiation scattering agents.

21. The composition of claim 20, wherein the lamellar encapsulated sunscreen active agent is a liposome-encapsulated sunscreen active agent.

22. The composition of claim 21, wherein the liposome is formed from soy lecithin.

23. The composition of claim 20, wherein the volatile additive is chosen from the group consisting of C₁-C₄ straight chain or branched chain alcohol, volatile silicone alcohol, C₁-C₄ volatile aldehyde and combinations thereof.

24. The composition of claim 23, wherein the volatile additive is ethanol.

25. The composition of claim 24 wherein the volatile additive is present in the composition in an amount less than 10% by weight.

26. The composition of claim 24 wherein the volatile additive is present in the composition in an amount between about 1% by weight and about 5% by weight.

27. The composition of claim 20, wherein the sunscreen active compound is chosen from the group consisting of para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum, 4-methylbenzylidenecamphor, and isopentyl 4-methoxycinnamate, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, and combinations thereof.

28. The composition of claim 20, wherein the UV-radiation scattering agent is a non-UV absorbing polymeric material.

29. The composition of claim 28, wherein the polymeric material comprises styrene/acrylates copolymers.

30. The composition of claim 29 wherein the polymeric material comprises spheres.

31. The composition of claim 20 further comprising additional cosmetic components.

32. The composition of claim 31, wherein the cosmetic components comprise one or more of self-tanning agents, insect repelling components, skin protectant active agents, absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, propellants, reducing agents, skin bleaching agents, skin-conditioning agents, skin protectants, solvents, foam boosters, hydrotropes, solubilizing agents, nonsurfactant suspending agents, waterproofing agents, and viscosity increasing agents.

33. The composition of claim 20, further comprising one or more additional skin anchoring components.

34. The composition of claim 33, wherein the one or more skin anchoring components is chosen from the group consisting of binding proteins, polysaccharides, glycoproteins, phospholipids, glycolipids, lipoproteins, lipopolysaccharide, lectins and combinations thereof.

35. The composition of claim 34, wherein the skin anchoring component comprises zinc pyridinethione.

36. A substantially waterproof, high SPF sunscreen composition comprising one or more lamellar encapsulated sunscreen active compounds in an aqueous dispersion containing one or more cosmetically acceptable volatile additivies, one or more UV-radiation scattering agents, and one or more skin anchoring components, wherein the composition provides an SPF on wet skin greater than the static SPF on dry skin.

37. The composition of claim 36, wherein the one or more skin anchoring components is chosen from the group consisting of binding proteins, polysaccharides, glycoproteins, phospholipids, glycolipids, lipoproteins, lipopolysaccharide, lectins and combinations thereof.

38. The composition of claim 37, wherein the skin anchoring component comprises zinc pyridinethione.

39. A liquid cosmetic composition for topical application to skin and/or hair comprising lamellar particles encapsulating at least one cosmetically effective benefit agent, a substantially aqueous continuous phase, one or more UV-radiation scattering agents and one or more cosmetically acceptable volatile additives, wherein the cosmetic composition provides a high SPF sunscreen.

40. A method of preventing erythema in a subject comprising topically applying to the subject's skin or hair a substantially aqueous composition comprising particles encapsulating at least one cosmetically effective benefit agent, a substantially aqueous continuous phase, and one or more cosmetically acceptable volatile additives, wherein the composition comprises a high SPF sunscreen.

41. The method of claim 40, wherein the composition further comprises one or more additional skin anchoring components.

42. The method of claim 41, wherein the one or more skin anchoring components is chosen from the group consisting of binding proteins, polysaccharides, glycoproteins, phospholipids, glycolipids, lipoproteins, lipopolysaccharide, lectins and combinations thereof.

43. The method of claim 42, wherein the skin anchoring component comprises zinc pyridinethione.

44. The method of claim 40, wherein the composition is applied prior to exposure to UV radiation.

45. A method of preventing photoaging of an object comprising applying to the surface of the object a substantially aqueous composition comprising particles encapsulating a UV-absorbing agent in a substantially aqueous continuous phase, one or more volatile additives, and one or more UV-radiation scattering agents.

46. A method of preventing photoaging of skin comprising applying to skin that will subsequently be exposed to UV radiation, the composition of claim 1.

47. A kit comprising a physiologically acceptable, dissolvable matrix comprising one or more lamellar encapsulated sunscreen active agents and one or more UV-radiation scattering agents and further comprising instructions to dissolve the matrix in a combination of water and at least one volatile additive so as to form the composition of claim 1.

48. The kit of claim 47, wherein the sunscreen active agents and the UV-radiation scattering agents are present in a form chosen from the group consisting of a powder and dry film.

49. The kit of claim 47 wherein the matrix comprises a woven or non-woven fabric substrate.

50. A kit comprising a plurality of zones, one of said zones comprising a mixture comprising one or more lamellar encapsulated sunscreen active agents and one or more UV-radiation scattering agents and another of said zones comprising at least one volatile additive, whereby the zones are physically separated from one another.

51. The kit of claim 50 comprising a plurality of zones separated by a breakable barrier, which barrier can be broken to form a continuous zone, to combine the components in the continuous zone.

52. A kit comprising a non-woven hydrophobic material comprising one or more lamellar encapsulated sunscreen active agents and one or more UV-radiation scattering agents and further comprising instructions to contact the material in a combination of water and at least one volatile additive so as to form the composition of claim 1.

53. A kit comprising a non-woven hydrophilic material comprising one or more lamellar encapsulated sunscreen active agents and one or more UV-radiation scattering agents and further comprising instructions to contact the material in a combination of water and at least one volatile additive so as to form the composition of claim 1.
